(19) United States
(12) Patent Application Publication
(10) Pub. No.: US 2021/0293779 A1
(43) Pub. Date: Sep. 23, 2021

(54) Method and Device for Actuating an Analysis Device for Running an Analysis on a Sample Material

(71) Applicant: Robert Bosch GmbH, Stuttgart (DE)

(72) Inventors: Jochen Rupp, Stuttgart (DE); Tom Doehring, Stuttgart (DE); Karsten Seidl, Mülheim an der Ruhr (DE); Tino Frank, Luzern (CH)

(21) Appl. No.: 17/047,632

(22) PCT Filed: Apr. 18, 2019
(86) PCT No.: PCT/EP2019/060055

§ 371 Date: Feb. 26, 2021

(30) Foreign Application Priority Data
Apr. 20, 2018 (DE) 10 2018 206 092.4

(51) Int. Cl. G01N 33/487; G16H 10/40

(52) U.S. Cl. CPC G01N 33/48792; G16H 10/40; G01N 33/48728; G01N 33/4875

(57) ABSTRACT

A method for actuating an analysis device for running an analysis on a sample material includes reading a user parameter, which represents an analysis to be carried out on the sample material, and loading a plurality of actuation commands for at least one analysis running unit of the analysis device from a command library. The method further includes actuating the at least one analysis running unit using the actuation commands in order to analyze the sample material.

### PRIOR ART

The invention is based on a device or a method according to the general type of the independent claims. A computer program is also an object of the present invention.

In vitro diagnostics (IVD) is a field of medical products that measure specific values (e.g. concentration of a molecule, presence of a specific then sequence, composition of blood) from human samples and enable a diagnosis and a decision as to treatment. This often takes place along a chain of multiple laboratory steps, wherein the sample is prepared in such a way that the target value can be measured without error. Various laboratory methods are applied, each of which has a device that is appropriate for the method. In point-of-care devices, the aim is to represent such in-vitro diagnostic tests in one device, and to reduce the number of the user's manual steps to a minimum. In the ideal case, the sample, i.e. the sample itself or the sample material, is placed directly into the (analysis) device, and the diagnostic test is processed fully automatically. Microfluidic systems—often referred to as lab-on-chip—are suitable in particular for processing and analyzing various biochemical diagnostic methods in a fluid state. Lab-on chip devices are as a rule developed or optimized for one measuring method (e.g. PCR, fluorescence measurement, pH measurement).

### DISCLOSURE OF THE INVENTION

Against this background, a method, furthermore a device that uses this method, and finally a corresponding computer program in accordance with the main claims, are introduced with the approach presented here. Through the measures described in the dependent claims, advantageous developments and improvements of the device given in the independent claim are possible.

To keep a lab-on-chip device as universal as possible, and to offer a general test platform, as many detection methods as possible should be combined with one another. Such a universal character, however, requires clearly defined interfaces in an extremely small physical space. In order to allow the greatest possible number of different tests to proceed on one platform, the machine or the analysis device should have the most universal possible control system that orchestrates individual components as effectively as possible for the application, and that exhibits a high degree of variation. The control system should also be as universal as possible so that reimplementation for new applications is not necessary. It should be possible to access an arsenal of existing commands and structures, and for only structures and commands that are missing to be implemented to supplement those that exist.

A method for actuating an analysis device for executing an analysis of a sample material is disclosed here, wherein the method comprises the following steps:
reading in a usage parameter that represents an analysis of the sample material that is to be run;
loading a plurality of actuation commands for at least one execution unit of the analysis device from a command library (for the execution unit); and
actuating the at least one execution unit making use of the actuation commands in order to analyze the sample material.

An analysis device in this context can refer to a device for the medical or biochemical analysis of samples. Sample material can, for example, refer to a piece of human or animal tissue or to a corresponding bodily fluid. A usage parameter can, for example, refer to information that is supplied manually or automatically to the analysis device and that specifies what type or kind of analysis is to be applied to the sample material. An actuation command can refer to machine-interpretable information or a control command regarding what activity or action an execution unit, as part of the analysis device or as a unit coupled to the analysis device, should run. Such an activity or action can, for example, be a processing of the sample material or the acquisition of a parameter of the sample material. A command library can, for example, refer to a command memory as a partial element of the analysis device. Alternatively or in addition, the command library can also be disposed either entirely or at least partially externally from the analysis device, for example as a part of a cloud computing network or on a memory of a container that contains the sample material.

The approach presented here is based on the recognition of the fact that an analysis of the sample material that is to be run requires highly varied activities or actions. Depending on the analysis to be run, different sample materials can be treated here in different ways, or a parameter thereof can be graded, wherein usually one or a plurality of execution units can be used as actuators that can perform a multiplicity of activities or actions with the sample material. The selection of a plurality of actuating commands from a command library thus makes it possible to perform a very flexible adaptation, or such a flexible use, of the execution unit for a very wide variety of analyses that are to be run. Depending on the specific use for an analysis that is specified by the usage parameter, the execution unit can now be actuated with an individual number or sequence of actuation commands in order to perform the analysis of the sample material to be run at the time.

The approach presented here thus offers the advantage of being able to perform an individual compilation of actuation commands from the command library for the execution unit of the analysis device, depending on what analysis of the sample material it is currently wished to run. In this way, the analysis device or the execution unit can be used in a very flexible manner, whereby the usage possibilities, and hence the application value of the analysis device or of the execution unit, can be correspondingly increased.

A form of embodiment of the approach proposed here is favorable in which, in the step of loading the plurality of actuation commands, the actuation commands are loaded from the command library as individual commands, and are linked into a command set appropriate for the analysis to be run, wherein the actuation step is carried out by using the command set. Such a form of embodiment of the approach proposed here offers the advantage that individual actuation commands that are to be run separately by the execution unit for the processing possibilities of the sample material can be individually compiled into the command set depending on the analysis that is currently required or to be run. In this way the execution unit can be actuated with actuation commands appropriate for the requirements of a temporal sequence of steps of processing the sample material for different analyses to be run, so that very high flexibility can be achieved in the usage of the execution unit.

A form of embodiment of the approach proposed here is furthermore advantageous in which actuation commands for multiple execution units are loaded in the loading step, wherein, in the actuation step, the multiple execution units are actuated making use of the actuation commands in order to analyze the sample material. Such a form of embodiment of the approach proposed here offers the advantage that, for example, individual execution units do not necessarily have to be designed in such a way that they have to be able to run all of the activities corresponding to the respective actuation commands. Rather the activities or working steps required according to the current analysis of the sample material that is to be performed can be executed or actuated in different execution units, whereby execution units that are of technically simpler design and that are thus more economical, or analysis devices of modular construction, can be employed for an economical analysis of the sample material.

According to a further form of embodiment of the approach proposed here, a step of storing an analysis result of an analysis of the sample material can be provided, wherein the analysis result is linked to data or metadata that represent an origin of the sample material, an analysis parameter and/or information relating to authorization to display or disseminate the analysis result. An origin of the sample material can, for example, refer to a name or an advice of the supplier of the sample material. An analysis parameter can, for example, refer to a physiological, biological or chemical parameter that was measured in the analysis of the sample material. Information relating to the authorization for a display or dissemination of the analysis result can, for example, be information that identifies that the output or dissemination of the analysis result may be suppressed and/or only made available for a specific questioner. Such a form of embodiment of the approach proposed here offers the advantage that disseminating or outputting the analysis result can be authorized very precisely, so that such information can already be combined or stored with the analysis result itself, and misuse of the analysis result can thus be reduced or, in the most favorable case, entirely prevented.

A form of embodiment of the approach proposed here in which a step is provided of entering at least one actuation command, which is unknown in the command library, for actuation of the at least one execution unit is furthermore particularly advantageous. Such a form of embodiment of the approach proposed here offers the advantage that, when manufacturing the execution unit, steps of processing a sample material by this execution unit that are possible but which have not yet been concretely specified can still be specified subsequently and can be stored in the command library in the form of a corresponding actuation command. In this way, the flexibility for the use of an already present execution unit in an analysis device can be further increased and, in some cases, analyses carried out whose individual steps of processing the sample material had not yet been disclosed during the development or manufacture of the execution unit or of the analysis unit.

A form of embodiment of the approach proposed here in which at least a heating, a movement, an illumination, a lighting, an exposure to sound and/or a sensing of a parameter of the sample material is carried out in the execution unit by at least one actuation command in the actuation step is furthermore advantageous. In particular, for carrying out an analysis of the sample material in the medical field, in particular in the for the acquisition of a physiological, biological or chemical parameter such as, for example, a viscosity of blood, the presence of an antigen after a vaccination or the like, the actuation of a corresponding processing of the sample material by the above-described working steps is particularly helpful, so that as a result the analysis in the execution unit of the analysis device can be performed in a very flexible and technically simple manner.

In order, for example, to satisfy statutory, official or economic requirements, according to a particularly favorable form of embodiment of the approach presented here a check can be carried out in the loading step as to whether an analysis of the sample material to be run by the user parameter in the analysis device can be carried out or may be carried out. In the loading step here, it is possible for no actuation commands to be loaded from the command library if the analysis of the sample material in the analysis device to be run by the user parameter cannot be carried out or may not be carried out.

A user parameter can, for example, refer to a parameter that supplies information regarding which user, for example a patient, a doctor or an official who is using the analysis device at the time, or the environmental scenario, for example a clinic, a medical practice or the home of a private user in which the analysis device is located at the time or is to carry out the analysis that is to be run. In this way it is for example possible to prevent an analysis which, in principle, can be carried out in the analysis device or the execution unit, being performed if this is not permitted or not desirable for particular reasons.

A particularly reliable and precise performance of the analysis can be ensured according to a further form of embodiment of the approach proposed here if, in the actuation step, the execution unit is actuated with the actuation commands depending on a parameter of the execution unit or a status of the analysis of the sample material that is to be run. The execution unit can, for example, be configured in such a way that a control command is not carried out until the sample material or the analysis procedure has reached a specific parameter or a specific criterion, for example has a predefined temperature. In this way, through taking this specific parameter or criterion into consideration, a performance of the analysis that is as free of errors as possible can be ensured through a precise control of the sequence of individual steps of the analysis by means of the actuation commands.

According to another form of embodiment of the approach proposed here, in the actuation step, the execution unit can be actuated by means of the actuation commands taking an intervention parameter into consideration, in particular wherein the intervention parameter is read in using a human-machine interface and/or using an automatically readable intervention parameter. Such a form of embodiment of the approach proposed here offers the advantage that an informed user can manipulate a flow of the analysis procedure in the execution unit or in the analysis device in order, for example, to bring about a restart of the analysis or to avoid damage to the analysis device or to the execution unit through an analysis that is not proceeding correctly. An automatically readable intervention parameter that is read, for example, from a container that contains the sample material, offers the advantage that the control of the flow of the partial analysis steps can be adjusted very precisely and in great detail for particular analyses that are to be performed.

A form of embodiment of the approach proposed here is particularly flexible in which a step of connecting the analysis device to a central computing unit is provided, wherein the computing unit is disposed externally to the analysis device and wherein, in the connection step, at least one actuation command is loaded from the central computer unit and/or an analysis result of the sample material is stored in the one central computing unit.

Such a form of embodiment of the approach proposed here offers the possibility of being able to access a large variety of actuation commands that have, for example, been preprogrammed by a plurality of users of the execution units or of the analysis devices. Alternatively or in addition, the analysis result can also for example be assessed by one or a plurality of external experts, without them having to come into the physical proximity of the analysis device or the corresponding execution unit.

According to a further form of embodiment at least one actuation command can be loaded in the loading step from a memory that is disposed outside the analysis device. In this way, a particularly advantageous compilation of actuation commands, or the selection of actuation commands for a particularly efficient execution of the analysis on at least one execution unit, can be performed for an analysis that is to be run.

This method can, for example, be implemented in software or hardware, or in a mixed form of software and hardware, for example in a control device.

The approach presented here further creates a device that is designed to carry out, actuate or implement the steps of one variant of a method presented here in corresponding apparatuses. The object underlying the invention can also be achieved quickly and efficiently through these variant embodiments of the invention in the form of a device.

The device can for this purpose comprise at least one computing unit for processing signals or data, at least one memory unit for storing signals or data, at least one interface to a sensor or to an actuator for reading sensor signals from the sensor or for outputting data or control signals to the actuator, and/or at least one communication interface for reading or outputting data that are embedded in a communication protocol. The computing unit can, for example, be a signal processor, a microcontroller or the like, wherein the memory unit can be a flash memory, an EEPROM or a magnetic memory unit. The communication interface can be designed to read in or output data wirelessly and/or via cables, wherein a communication interface that can read in or output wired data can read these data, for example electrically or optically, from a corresponding data transmission line or output them into a corresponding data transmission line.

A device can in this context refer to an electrical device that processes sensor signals and outputs control and/or data signals depending thereon. The device can comprise an interface that can be implemented through hardware and/or software. In a hardware implementation, the interfaces can for example be part of what is known as a system ASIC that contains a wide variety of functions of the device. It is, however, also possible for the interfaces to be their own, integrated circuits, or consist at least partially of discrete components. In a software implementation, the interfaces can be software modules which are present, for example, on a microcontroller in addition to other software modules.

A computer program product or computer program with program code that can be stored on a machine-readable carrier or storage medium such as a semiconductor memory, a hard disk memory or an optical memory, and which is used for carrying out, implementing and/or actuating the steps of the method according to one of the forms of embodiment described above in particular when the program product or program is carried out on a computer or device is also advantageous.

Exemplary embodiments of the approach presented here are illustrated in the drawings, and explained in more detail in the following description. Here:

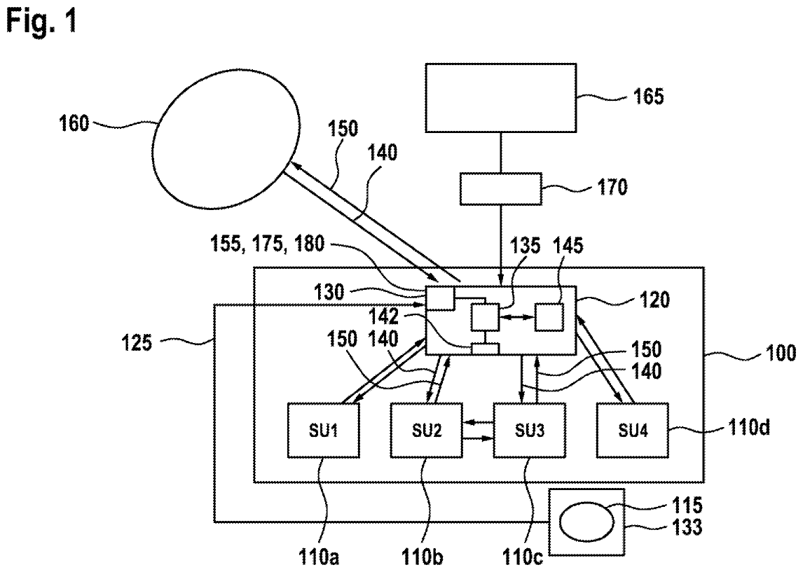

FIG. 1 shows a block diagram of an exemplary embodiment of an analysis device;

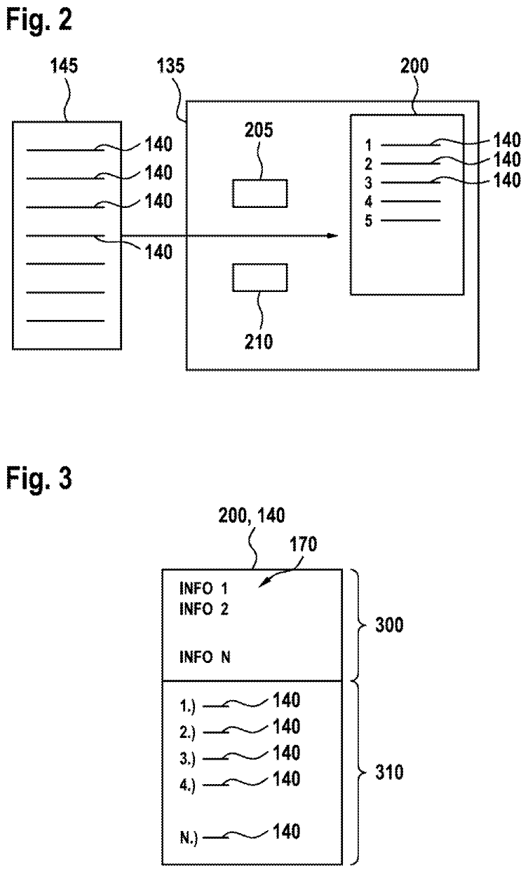

FIG. 2 shows a schematic illustration of the structure and the mode of operation of the loading unit with a protocol library;

FIG. 3 shows a schematic illustration of a general structure of the protocol or command set;

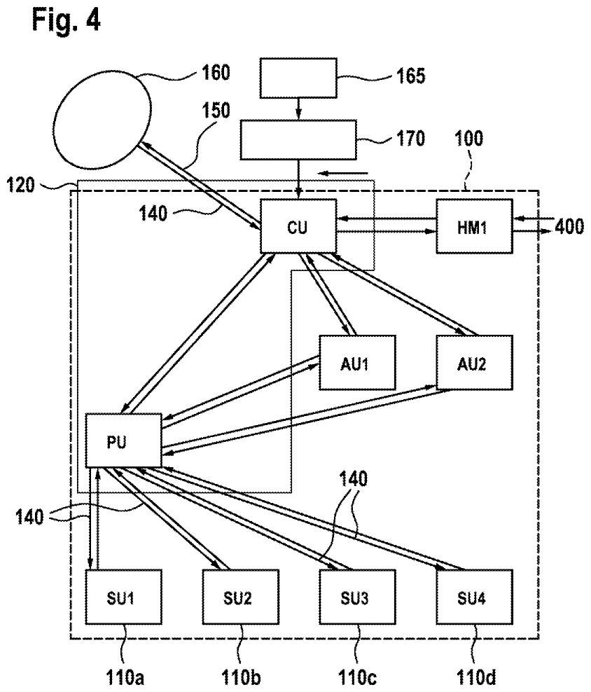

FIG. 4 shows a block diagram of an extended, universal system of a further exemplary embodiment of an analysis device;

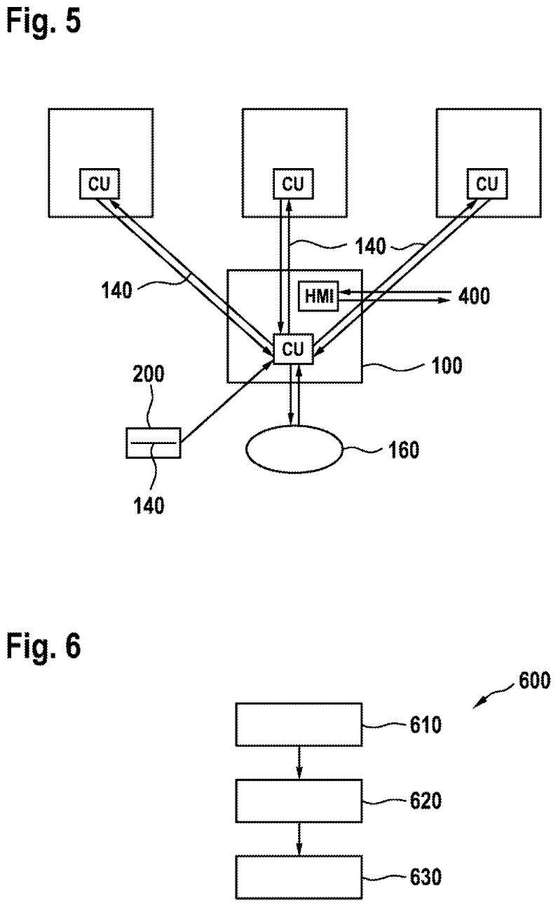

FIG. 5 shows a schematic block diagram illustration of a further exemplary embodiment of an analysis device; and

FIG. 6 shows a flow diagram of a method according to an exemplary embodiment.

In the following description of favorable exemplary embodiments of the present invention, the same or similar reference signs are used for elements shown in the various figures and having similar effects, repeated description of these elements being omitted.

FIG. 1 shows a block diagram of an exemplary embodiment of an analysis device 100 in which a plurality of execution units 110a, 110b, 110c and, 110d are provided, which can also be referred to as subunits SU1 to SU4.

These execution units 110a to 110d are, for example, designed to accept a sample material 115 that is, for example, composed of a human or animal tissue, a corresponding bodily fluid or the like, and process it in accordance with a predetermined processing specification in order to obtain two of its parameters of this sample material 115. The execution units 110a to 110d can, for example, be designed to heat the sample material, to shake it thoroughly, to illuminate it, to subject it to sound, to mix it with or bring it into contact with an analysis liquid such as, for example, alcohol or an enzyme in order to process the sample material 115 for determining the desired parameter. The execution units 110a to 110d can also be designed not to perform a plurality of different actions in relation to the sample material 115, but for each of the execution unit 110a to 110d to run a specific activity or actions in relation to the sample material 115, wherein the sample material 115, or the appropriately treated sample material 115, is then transferred from one execution unit, for example the execution unit 110b, to a further execution unit 110c, where it is further processed and, for example, transferred back again to the execution unit 110b. A flexible analysis of the sample material 115 can in this way take place in economical execution units 110.

In order to be able now to perform the analysis of the sample material 115 by means of the analysis device 110, the analysis device 110, or the corresponding execution units 110a to 110b, is or are designed to be able to perform the most varied possible analysis procedures with the same execution units 110a to 110d, in order to enable the most flexible possible use in different application scenarios. For this purpose it is however necessary for the individual execution unit 110a to 110d, or a combination of multiple execution units 110a to 110d to be actuated with appropriate actuation commands in order to be able to perform correctly the process steps necessary in the individual execution units 110a to 110d for the analysis of the sample material 115 that is currently desired. If the analysis device 110 is designed such that many different process steps can be run on the execution units 110a to 110d (also for example in a freely selectable sequence), it is now of central importance to be able to perform the selection of the individual process steps in an appropriate control device 120. The approach presented here comes into play at this point, serving to make the actuation of one or a plurality of execution units 110a to 110d for the analysis of the sample material 115 that is to be run in each case highly flexible.

In order to be able to make the actuation of the execution units 110a to 110d highly flexible as described above, a usage parameter 125 is first read in via a reading interface 130 in the control device 120. The usage parameter 125 can, for example, be a code on a container 133 in which the sample material 115 is disposed, for example a laboratory chip in which a drop of blood is held. Through this code or usage parameter 125, the control device 120 can, for example, be informed that an analysis of the drop of blood as sample material 115 is now to be run. This usage parameter 125 can then be transmitted in the control device 120 to a loading unit 135 that ascertains the sequence of actuation commands 140 for one or a plurality of execution units 110a to 110d that correspond, for example, to predefined actuation commands 140 that are stored in a command library 145. Each of these actuation commands 140 here corresponds to information for one of the execution units 110a to 110d to perform a specific activity or actions in relation to the sample material 115 (or a correspondingly processed sample material 115). The actuation commands 140 can then be transmitted as a command set by means of an actuation unit 142 to the relevant execution units 110a to 110d that process the sample material 115 in accordance with these actuation commands 140 in order to analyze the sample material 115. As was already described above, individual analysis steps or partial analysis steps can also here be carried out in different instances of the execution units 110b or 110c, in particular when the relevant execution unit 110b or 110c is (for example for reasons of cost or for technological reasons) only implemented in such a way that only particular partial analysis steps can be carried out on the respective execution unit 110b or 110c.

The command library 145 can here be stored on a memory element of the analysis device or of the control device 120 itself. Alternatively or in addition, a command library 145 that is entirely or partially disposed outside the control device 120 or the analysis device 110, can also be used. The command library can, for example, also be disposed or stored entirely or partially in a memory of a container 133 that contains the sample material 115, or stored in a computer network that is configured as a cloud server.

An analysis result 150 or partial analysis result that is for example obtained from the execution unit 110b or 110d concerned at the time after carrying out the partial analysis steps brought about by the corresponding actuation commands 140, can then, for example, be transmitted back to the control device 120. A decision can then, for example, be made in the control device 120, for example on the basis of further parameters such as, for example, a user parameter 155 that is read in via the reading interface 130, as to whether the analysis result 150 can or may be displayed or, for example, transmitted to an external computing unit 160 that is configured as a cloud computer. Actuation commands 140, which for example are made available subsequently by the manufacturer of the execution units 110a to 110d or preprogrammed by other users of types of the execution units 110a to 110d, can, for example, also be read from the external computing unit 160 into the control device 120.

In order now to be able to use the execution units 110a to 110d as flexibly as possible and, for example, also to be able to run partial analysis steps or a sequence of analysis steps that were not provided during the manufacture of the execution units 110a to 110d, an input interface 165 can furthermore also be provided through which, for example, a protocol 170 that contains one or a plurality of “new” actuation commands 140 is provided to the analysis device 110, so that this “new” actuation command 140 can be stored in the command library 145. In this way the use of the analysis device or of the respective execution units 110a to 110b can be made even more flexible.

In addition or alternatively a location parameter 175 that represents a usage environment of the analysis device 100 can, for example, also be read in through the reading interface 130. The location parameter 175 can, for example, indicate that the analysis device 100 is located in a hospital, a medical practice or privately at the home of a user, so that, for example, specific analyses which technically could be carried out on the basis of actuation commands 140 present in the command library 145 but which however may not or should not be carried out in the specific application environment at which the analysis device 100 is currently located are indeed not carried out. In this context it is, for example, possible to prevent an analysis of the sample material 115 for the presence of a highly infectious virus from being carried out if the location parameter 175 indicates that the analysis device is located in a medical practice, since such an analysis for the presence of a virus of that type should be restricted to a hospital set up with the presence of suitable safety laboratories or an appropriate research facility. In this way, in the case of a correspondingly positive analysis result, the risk of persons being infected by the sample material 115 can be reduced by the largest possible extent.

An intervention parameter 180 that represents intervention on the part of a user in the execution of the analysis or the sequence in which the actuation commands are carried out can also be read in through the reading interface 130. The user can, for example, by means of the intervention parameter 180 that is read in, for example, via a human-machine interface HMI to be described in more detail below, be used to bring about an interruption in the execution of the partial analysis steps that are actuated by the actuation commands 140 in the execution units 110a to 110d. It is, however, also conceivable that the intervention parameter 180 can be fetched up as a specific item of information for control of the workflow from a data carrier, in particular a passive data carrier, (e.g. an RFID) in the single-use cartridge serving as a container 133 of the sample material 115. In this way, specific parameters for the actuation command or commands, or for the signal evaluation and the control device 120, or for a relevant execution unit 110, can for example also be transmitted. Parameters related to specific reagents, reagent batches or individual cartridges or cartridge batches are particularly of interest here. Partial commands, complete commands or command chains, as well as metainformation can however also be stored on the data carrier of the cartridge and appropriately fetched up and processed by the analysis device 100 or by one or a plurality of the execution units 110. The use of the RFID as a carrier of the intervention parameter 180 also offers the possibility of marking the cartridge as used after it has been used through what is known as a kill switch (for example in the form of an irreversibly settable flag) in order to thus prevent reuse.

One aspect of the approach presented here can thus be seen in a control system or a control device 120 that orchestrates general system components or execution units 110a to 110d of a microfluid platform for assays, i.e. the analysis device 100, in a specific manner and which enables easy system integration of new assays (which can here also be referred to as analyses) and applications. The control system 120 also allows control of a plurality of platform analyzers or execution units 110a to 110d via a common network or a cloud 160.

An important aspect of the approach presented here can be seen in an interface protocol that monitors or controls hardware components such as the execution units 110a to 110b through a general command library 145 in the temporal sequence. The command library 145 is designed in such a way that new (actuation) commands 140 can be added easily. The dependencies of the commands 140 are regulated by a rule and specification library that is, for example, implemented as part of the command library 145 and that is consulted as the actuation commands 140 are read out for consistency of the actuation commands 140 or of the sequence of actuation commands 140 for the respective relevant execution unit 110a to 110d.

Exemplary embodiments of the approach presented here entail the following advantages:
1) There is uniform control of all installed system components 110a to 110d, and they can be adapted individually to individual applications through a universal command library 145.
2) The command structure makes it possible to realize a universal platform as an analysis device 100 with a simple facility for implementing more commands 140.
3) New elements such as, for example, further execution units 110 can easily be integrated through interfaces.
4) The control of a wide variety of elements or execution units 110 is possible on different hierarchical levels. An analyzer or an analysis device 100, or also however a system of analyzer or analysis devices or execution units 110, can be monitored or controlled.
5) Similar application or partial analysis steps can be implemented in a simplified manner through simplified inheritance of the actuation commands 140.
6) Quality standards can be implemented through an inherent rulebook, requirements and control functions implemented in the control device 120.
7) The protocol of the sequence of actuation commands 140 that is employed makes it possible to test whether the hardware in use, such as the execution units 110, is adequate for the test or analysis requirements being used. This is important inasmuch as different hardware versions of devices or execution units 110 are in use in the field. It is also possible in this way to ensure that tests or analyses that are only permitted to central laboratories are not carried out on a machine or an analysis device 100 that is, for example, carried out in a medical practice where the test or the analysis concerned is not permitted. This control is seen as an important part of the fleet management for the utilization of analysis device 100.
8) A human interface can be integrated universally, and the application adapted so that a user can, for example, also influence an analysis through interaction with the analysis device 100 or the control device 120.
9) The use of conditional functions permits dynamic control of applications or analyses. No fixed parameters have to be measured, but rather it is possible for an (analysis) step for example not be carried out until a certain condition of the sample material or of an element of the execution unit 110 is reached. A system or an analysis device 100 can be adapted in this way to environmental conditions. The temperature may be mentioned as an example here, wherein a partial analysis step in one of the execution units 100 is for example not carried out until the sample material 115, or a component of the execution unit 110 in which a relevant partial analysis step is to be carried out, has a specific temperature required for the successful implementation of the partial analysis step. A temperature-sensitive step is then, for example, not carried out until the appropriate temperature has been reached.
10) The control system, or the control device 120 with its rulebook and general command library 145 accelerates the development of new applications or analyses that can be carried out on the analysis device 100.

An exemplary embodiment of the approach presented here is described once more in more detail below. The schematic structure and the monitoring mechanism for the system or analysis device 100 is illustrated in FIG. 1. This is a microfluid analyzer as the analysis device 100, which comprises a plurality of partial components as execution units 110 (SU, subunits) that are, for example, designed as pneumatic elements, heaters, cameras, filters, light sources or a sound source. All these components 110 are to be actuated and monitored at the correct time for a microfluid assay or an analysis. To ensure that this occurs smoothly, they are controlled by a control unit or the control device 120.

The subunits (which can also be referred to synonymously as execution units 110) can receive signals (as the actuation commands 140) from the control unit (also known synonymously as the control device 120) as well as transmit data (for example the analysis result 150). The control unit 120 can furthermore also communicate with an external cloud 160. In order to use the analyzer or the analysis device 100 in the most universal possible way, a protocol, for example, containing general commands for control of the control unit 120, is loaded into the control unit 120. This converts the commands 140 appropriately into the language of the subunits 110 and controls each of them.

The protocols can be prepared with the aid of a protocol generator implemented in FIG. 1 as in the loading unit 135. In addition to the interface to the library (that is also referred to synonymously as the command library 145) of permitted commands 140 (i.e. actuation commands), this contains a rulebook as to how commands 140 can be linked and a requirements list for the preparation of new commands 140.

The control unit 120 is alone responsible for the coordination and actuation of the individual subunits 110. Even if a new subunit 110 is to be installed in the analyzer 100, this is monitored via the interface of the control unit 120.

FIG. 2 shows a schematic illustration of the structure and the mode of operation of the loading unit 135 with a protocol library 200. The heart of this structure is the use of the library 145 as a collection of permitted and tested commands 140 for the analyzer 100 or the corresponding execution units 110. The commands 140 are ordered according to the mode of operation of the individual subunits 110. This library 145 is universal, and arbitrary sequence programs, i.e. protocols or command sets 200 can be compiled from these commands 140. In order to implement a sequence program, i.e. a command set 200 that successfully carries out a particular application or analysis, the library 145 is accompanied by a rulebook 205 and a requirements book 210. These rules 210 describe how which commands 140 can be combined into a successful sequence of the analysis. The requirements 210 ensure the permitted parameters and conditions (e.g. temperature, step length etc. . . . ) that crystallize out of the requirements of the machine or execution unit 110 and its software components. The library 145 (or, more precisely, the commands 140 stored therein) is ideally written in an extended markup language (e.g. XML, SGML, RSS) that makes it possible for new commands to be added successively to the library 145 while giving consideration to the requirements 210 and the rulebook 205. This permits a simple, ongoing extension of the universal command library 145 as an integral component of a universal analyzer platform or of the analysis device 100. The protocols 200 are written in an extended markup language, while the processing is integrated into the hardware of the analyzer 100.

FIG. 3 shows a schematic illustration of a general structure of the protocol or command set 200. This consists in general of two parts. A first part 300 forms a global information part which the metainformation that can be called up at any time during the progress of an assay. This contains, for example, which assay or which analysis for performance on the execution units 110 is concerned, patient information, machine requirements information and information that can be displayed to the user of the analysis device 100. This information can also be exploited in the first part 300 in order to check whether the machine or the execution unit 110 being used corresponds to the requirements for the corresponding analyzer 100. This is of interest in the case of medical devices to the extent that the same analyzer 100 may be located in an analysis laboratory, or with a general medical practitioner as a patient-near analyzer, or on a clinical ward. These have different permissions. An analyzer 100 in a medical practice certainly would have the capacity for carrying out a test or an analysis whose performance is only valid or permitted in an analytical laboratory. This would, however, be forbidden from a regulatory perspective. The location in a location parameter 170, or a hardware revision (e.g. for which analyses the analysis device 100 is permitted and for which it is not), can then be compared with the metainformation in the first part 300 of the command set 200, and a possible misuse can be prevented. An assay or an analysis could also require specific hardware components as the execution unit 119 in the analyzer 100, which are only specifically built into a certain analyzer 100. The presence of these components 100 can be ensured through the metainformation in the first part 300.

The second part 310 comprises for example a section of temporary step information that is implemented as actuation commands 140 for the relevant execution unit 110 or for a plurality of execution units 110. These steps or commands 140 are processed successively in an ordered sequence in defined time steps by the execution unit or execution units 110. The actual formulation, or the command set 200 of the application assay, i.e. the analysis to be run, is thus implemented. The commands 140 that actuate the correct universal subunits 110 at the correct time in the correct context are taken for this purpose from the library 145. The requirements 210 and the rules 205 again here establish the framework, and provide support in the correct application. When time steps are not clear from the beginning, conditions can also be implemented as criteria to be satisfied. A subsequent command 140 is not carried out here until a certain value within the system (i.e. of the analysis device 100 or at least of one of the execution units 110) is reached. The temperature may be mentioned here as an example. A certain step only begins when a subunit 110 designed as a heater has reached a certain temperature value. The general signal traffic between the subunit 110 and the control unit or the control device 120, which has a universal implementation and is applied in an application-specific manner here, is utilized.

FIG. 4 shows a block diagram of an extended, universal system of a further exemplary embodiment of an analysis device 100. The control device 120 here can comprise a plurality of subunits. The protocol is, for example, handled by a control unit CU of the control device 120. This in turn is connected to a plurality of subunits. A processing unit PU is installed here, and controls fundamental operative subunits such as heaters, optical equipment, pneumatics and so forth. This processing unit PU has its own internal controller, and preprocessing facilities for information and commands are already integrated.

This enables fast processing and orchestration of the operative subunits such as the execution units 110a to 110d and an initial, rapid evaluation of the sample material or of the analysis result. These steps are permanently implemented, and can be actuated via the protocol using defined commands 140. This simplifies implementation of the protocol. Since in many commands 140 certain fundamental units can operate in parallel, this allows a faster processing of the specification for carrying out the analysis. For the majority of applications the operative commands 140 are themselves equivalent in a universal platform as an analysis device 100. Evaluation and data processing, on the other hand, often require an application-specific data pipeline and evaluation chain. For that reason, in addition to the processing unit PU, additional evaluation units AU1 or AU2 (AU=analytical unit) are also implemented. Actuation for these can be added as a software package in a modular manner via interface definition and new protocol commands 140. The transfer is, for example, regulated in the global part of the protocol 200.

A further aspect of an exemplary embodiment of the approach presented here can be seen in the connection of a human-machine interface, HMI. This allows the actual user 400 to interact with the machine or the analysis device 100. The control unit CU of the control device 120 can thus continuously test whether the protocol or the actuation commands 140 should, for example, be handled as a command set 200, or whether the user 400 can manipulate the protocol or the procedure for carrying out the actuation commands 140, in particular wishes to interrupt the protocol or the execution of the analysis. User interactions are also necessary in certain applications or during the execution of analyses on the analysis device 100. This is achieved through connecting the user 400 by means of the human-machine interface HMI. What data and results are displayed on a screen is also regulated through the connection of the user by means of the human-machine interface HMI. The global metainformation in the protocol 200 makes the connection of the user 400 via the human-machine interface HMI application-specific.

FIG. 5 shows a schematic block diagram illustration of a further exemplary embodiment of an analysis device 100, and its connection to further components. The same construction involving the use of actuation commands 140 can be employed here so that a plurality of analyzers or analysis devices 100 can be networked to one another, which can for example take place via a dedicated network or via a cloud 160. The CU of one analyzer 100 is here used as the primary CU that coordinates the processes or analysis steps of the other CUs so that the overall process proceeds successfully. The human-machine interface HMI is also controlled by this CU.

FIG. 6 shows a flow diagram of an exemplary embodiment of the present invention as a method 600 for actuating an analysis device for carrying out an analysis of a sample material. The method 600 comprises a step 610 of reading in a usage parameter that represents an analysis of the sample material that is to be run. The method 600 further comprises a step 620 of loading a plurality of actuation commands for at least one execution unit of the analysis device from a command library for the execution unit. Finally, the method 600 comprises a step 630 of the actuation of at least one execution unit making use of the actuation commands in order to analyze the sample material.

If an exemplary embodiment comprises an “and/or” combination between a first feature and a second feature, this is read such that the exemplary embodiment according to one form of embodiment exhibits both the first feature as well as the second feature and, according to a further form of embodiment, either only the first feature or only the second feature.

1. A method for actuating an analysis device for executing an analysis of a sample material, comprising:
reading in a usage parameter that represents the analysis of the sample material;
loading a plurality of actuation commands for at least one execution unit of the analysis device from a command library; and
actuating the at least one execution unit using the actuation commands to analyze the sample material.

2. The method as claimed in claim 1, wherein:
the loading of the plurality of actuation commands includes loading the plurality of actuation commands from the command library as individual commands, and linking the plurality of actuation commands into a command set configured for the analysis to be run;
the actuation of the at least one execution unit includes using the command set.

3. The method as claimed in claim 1, wherein:
the loading of the plurality of actuation commands includes loading actuation commands for a plurality of execution units; and
the actuation of the at least one execution unit includes actuating the plurality of actuation units using the loaded actuation commands to analyze the sample material.

4. The method (600) as claimed in claim 1, further comprising:
storing an analysis result of the analysis of the sample material,
wherein the analysis result is linked to metadata that represent an origin of the sample material, an analysis parameter, and/or information relating to authorization to display or disseminate the analysis result.

5. The method (600) as claimed in claim 1, further comprising:
entering at least one actuation command unknown in the command library for actuating the at least one execution unit.

6. The method as claimed in claim 1, wherein the actuation of the at least one execution unit includes including executing at least a heating, a movement, an illumination, a lighting, an exposure to sound, and/or a sensing of a parameter of the sample material in the execution unit.

7. The method as claimed in claim 1, wherein the loading of the plurality of actuation commands includes:
performing a check as to whether the analysis of the sample material can be carried out in the analysis device based on a location parameter; and
if the check determines that the analysis of the sample material cannot be carried out in the analysis device, no actuation commands are loaded from the command library.

8. The method as claimed in claim 1, wherein the actuation of the at least one execution unit includes actuating the at least one execution unit based on a parameter of the execution unit or a status of the analysis of the sample material to be run with the actuation commands.

9. The method as claimed in claim 1, wherein the actuation of the at least one execution unit is performed based on the actuation commands and an intervention parameter.

10. The method as claimed in claim 1, further comprising:
connecting the analysis device to a central computing unit that is disposed externally to the analysis device, the connecting including loading at least one actuation command from the central computing unit and/or storing an analysis result of the sample material on the central computing unit.

11. The method as claimed in claim 1, wherein the loading of the plurality of actuation commands includes loading at least one of the plurality of actuation commands from a memory that is disposed outside the analysis device.

12. A device configured to actuate an analysis device to execute an analysis of a sample material, comprising:
a reading interface configured to read in a usage parameter that represents the analysis of the sample material;
a loading unit configured to load a plurality of actuation commands for at least one execution unit of the analysis device from a command library; and
an actuation unit configured to actuate the at least one execution unit using the actuation commands to analyze the sample material.

13. A computer program configured to be executed to actuate an analysis device for executing an analysis of a sample material, the computer program configured to:
read in a usage parameter that represents the analysis of the sample material;
load a plurality of actuation commands for at least one execution unit of the analysis device from a command library; and
actuate the at least one execution unit using the actuation commends to analyze the sample material.

14. The computer program as claimed in claim 13, wherein the computer program is stored on a machine readable storage medium.

15. The method as claimed in claim 9, wherein the intervention parameter is read in using a human-machine interface (HMI) and/or is an automatically readable intervention parameter.
